(19) United States
(12) Patent Application Publication
(10) Pub. No.: US 2025/0037812 A1
(43) Pub. Date: Jan. 30, 2025

(54) SYSTEMS AND METHODS FOR IDENTIFYING SUBJECTS FOR CLINICAL TRIALS

(71) Applicant: Eli Lilly and Company, Indianapolis, IN (US)

(72) Inventors: Antoniu Laurentiu FANTANA, Bolton, MA (US); Klaus Theodor GOTTLIEB, Cayucos, CA (US); William Robert PRUCKA, Zionsville, IN (US)

(21) Appl. No.: 18/713,758

(22) PCT Filed: Nov. 16, 2022
(86) PCT No.: PCT/US22/50020

§ 371 Date: May 28, 2024

Related U.S. Application Data
(60) Provisional application No. 63/284,345, filed on Nov. 30, 2021.

(51) Int. Cl. G16H 10/20; G16H 15/00

(52) U.S. Cl. CPC G16H 10/20; G16H 15/00

(57) ABSTRACT

A computing device is provided having a processor in communication with a memory configured to store machine-readable instructions. The processor can access data indicative of times of a plurality of healthcare visits of the patient over time during an observation period, determine a metric based on the times of the plurality of healthcare visits of the patient over time during the observation period, and identify a patient for inclusion in the clinical trial when the metric exceeds a metric threshold.

### BACKGROUND

Many chronic diseases are associated with disease flares. A disease flare is a measurable increase in disease activity, which can present as new or worsening symptoms, clinical signs, or laboratory measurements. Disease flares are typically temporary, ranging from a few hours to a few weeks or months. After a disease flare, the associated conditions can become dormant until another flare occurs. It can be desirable to enroll patients undergoing disease flares into clinical trials. However, oftentimes patients may only qualify for a clinical trial when they are experiencing a disease flare, since the clinical trials can be subject to strict inclusion and exclusion criteria. For example, the symptoms, clinical signs, and/or laboratory measurements of a patient who is not experiencing a disease flare may not fit the criteria necessary for inclusion in a clinical trial (e.g., since the patient may not provide useful data for the trial). Since disease flares can be unpredictable, clinical trial recruitment can be challenging.

### SUMMARY

The present disclosure relates to techniques for identifying patients for inclusion in a clinical trial based on the patient's history of healthcare visits. In particular, the present disclosure relates to assessing the timing of the patient's healthcare visits to determine a degree of irregularity of the patient's healthcare visits. The degree of irregularity can be used to determine when a patient may be undergoing a disease flare (e.g., and thus be used as an indicator for a patient's inclusion into a clinical trial).

In one embodiment, the techniques provide for a computerized method for identifying a patient for inclusion in a clinical trial. The method includes accessing data indicative of times of a plurality of healthcare visits of the patient over time during an observation period. The method includes determining a metric based on the times of the plurality of healthcare visits of the patient over time during the observation period. The method includes identifying the patient for inclusion in the clinical trial when the metric exceeds a metric threshold.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a block diagram of an illustrative system 100 for identifying a patient for inclusion in a clinical trial, according to some embodiments.

FIGS. 2A-B are flowcharts showing exemplary computerized methods for identifying a patient for inclusion in a clinical trial, according to some embodiments.

FIG. 3A is a chart illustrating regularly spaced healthcare visits, according to some embodiments.

FIG. 3B is a chart illustrating irregularly spaced healthcare visits, according to some embodiments.

FIG. 4 shows an illustrative implementation of a computer system that may be used to perform any of the aspects of the techniques and embodiments disclosed herein, according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

Provided herein are software-based techniques for identifying a patient for inclusion in a clinical trial. According to some embodiments, the techniques can evaluate data indicative of a pattern of healthcare visits of a patient over time. For example, the techniques can evaluate the spacing, or time intervals, between healthcare visits of the patient over a period of days, weeks, months, years, etc. According to some embodiments, relatively regular time intervals may indicate that a patient is receiving continuous, scheduled care (e.g., chemotherapy treatments). Conversely, relatively irregular time intervals (i.e., wherein the healthcare visits are relatively clustered in time) may indicate that the patient is receiving episodic care (e.g., emergency room visits). According to some embodiments, the techniques can determine the degree of irregularity (or regularity) of the timing of healthcare visits of the patient and identify the patient for inclusion in a clinical trial if the degree of irregularity exceeds a threshold. For example, a patient who is receiving a relatively high degree of irregular care may be identified for inclusion in the clinical trial.

The inventors have appreciated the importance of clinical trial enrollment for advancing treatment of chronic diseases. Not only does patient enrollment help to advance a new treatment towards FDA approval, but it can also make the treatment immediately available to that patient. However, the inventors have further appreciated that it can be challenging to enroll patients for clinical trials related to treatment of chronic disease. As described herein above, some chronic diseases are associated with disease flares, or temporary, measurable increases in disease activity. Due to the inclusion and exclusion criteria of some clinical trials, chronic disease patients may not be eligible for enrollment unless they are experiencing a disease flare at the time of their screening visit. Due to these enrollment challenges, clinical trials may be delayed, with some enrollment sites never enrolling a single patient. Furthermore, the inventors have appreciated that it can be challenging to detect disease flares in patients with chronic disease, increasing the challenge of patient enrollment. Conventional techniques for detecting disease flares include using algorithms (e.g., machine learning models) to evaluate clinical notes and Medicare claims. The inventors have appreciated that such conventional techniques are typically both complex and disease specific, making them difficult to implement and, once implemented, inapplicable to most chronic diseases except for the specific disease for which the techniques were developed. Furthermore, disease flares associated with some chronic diseases are not well-documented, making it challenging to use such conventional techniques, which rely on clinical documentation for accurate performance.

Accordingly, the inventors have developed software-based techniques to identify patients for inclusion in a clinical trial that provide improvements to conventional technology used to evaluate patients for clinical trials. In some embodiments, the techniques can evaluate a pattern of the patient's healthcare visits over time. The inventors have appreciated that the pattern of a patient's healthcare visits may provide a useful indication of the patient's condition or a status of the patient's health. For example, if a patient is visiting a healthcare facility regularly (e.g., at evenly spaced time intervals), then this may indicate that they are receiving regular treatments and/or medical exams for some condition (e.g., a chronic disease). Conversely, if a patient is visiting a healthcare facility irregularly (i.e., the patient's visits are relatively clustered in time), then it may indicate that they are experiencing new or worsening symptoms related to a condition. The inventors have appreciated that this may be helpful for identifying a patient with chronic diseases who is experiencing a disease flare. For example, a patient with arthritis may visit a healthcare facility more frequently when they are experiencing pain and less frequently when the pain subsides. According to some embodiments, the techniques can determine a metric that measures a degree of irregularity or clustering of the timing of the patient's healthcare visits. The inventors have appreciated that many people visit healthcare facilities with some degree of irregularity, however, quantifying the degree of irregularity may help to distinguish patients experiencing heightened disease conditions (e.g., a disease flare) relative to other people. Accordingly, in some embodiments, the techniques can use the metric indicative of the degree of irregularity to identify a patient who is experiencing a disease flare and/or for inclusion in a clinical trial. For example, in some embodiments, the techniques compare the metric to a threshold, and identify the patient for inclusion in a clinical trial when the metric exceeds the threshold. As described herein, the techniques do not require training or deploying complex machine learning models. Accordingly, the techniques described herein can be implemented faster and easier than conventional techniques and can be used to analyze a plurality of different diseases. Further, since the techniques described herein do not rely on training data that may not be understood and/or available (e.g., clinical documentation), the techniques described herein can be deployed when conventional techniques may not otherwise be able to be deployed (e.g., due to a lack of training data and/or understanding of the data).

The inventors have further appreciated that the software-based techniques may be useful for developing treatment plans or interventions for patients with chronic diseases. In some embodiments, based on the degree of irregularity of the timing of the patient's healthcare visits, the techniques can identify whether the patient should be receiving regular, continuous care, as opposed to irregular, episodic care. For example, irregular, episodic care may be most appropriate for patients having conditions that are one-off episodes and that have a foreseeable endpoint. Typically, when such patients reach the endpoint, they no longer require care for that condition. Conversely, patients who have a complex, high-risk, chronic disease may benefit from regularly scheduled, continuous care. The inventors have appreciated that the techniques may help healthcare providers to educate and provide interventions for patients who are diagnosed with a chronic disease but are only receiving episodic care. For example, a doctor in the emergency room can use the techniques described herein to identify whether such a patient is visiting the healthcare facility with a high degree of irregularity (e.g., above a threshold.) If so, the doctor may provide the patient with resources for seeking continuous, long term care.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

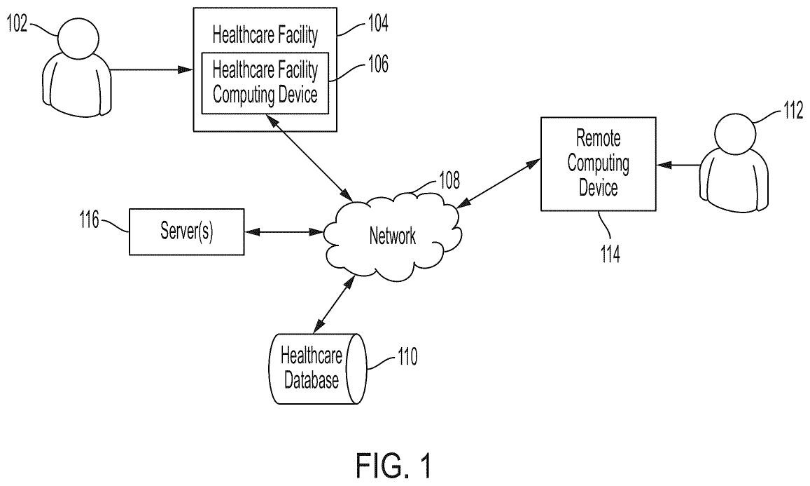

FIG. 1 is a block diagram of an illustrative system 100 for identifying a patient for inclusion in a clinical trial, in accordance with some embodiments. In the illustrative example of FIG. 1, a patient 102 may visit healthcare facility 104. Data related to the patient's visit to the healthcare facility 104 may be provided to healthcare facility computing device 106, which may be communicatively coupled to healthcare database 110, remote computing device 114, and server 116. It should be appreciated that system 100 is illustrative and that a system may have one or more other components of any suitable type in addition to or instead of the components illustrated in FIG. 1. For example, there may be additional remote systems (e.g., two or more) present within a system.

Network 110 may be or include a wide area network (e.g., the Internet), a local area network (e.g., a corporate Internet), and/or any other suitable type of network. Any of the devices shown in FIG. 1 may connect to the network 110 using one or more wired links, one or more wireless links, and/or any suitable combination thereof. Accordingly, the network 110 may be, for example, a hard-wired network (e.g., a local area network within a healthcare facility), a wireless network (e.g., connected over WiFi and/or cellular networks), a cloud-based computing network, or any combination thereof.

In the illustrative embodiment of FIG. 1, the at least one healthcare database 110 may store data indicative of visits of the patient 102 (and/or one or more other patients) to the healthcare facility 104. The data may be indicative of the timing of healthcare visits of the patient 102, medical history data for the patient 102, test result data for the patient 102, and/or any suitable data for the patient, as aspects of the technology described herein are not limited in this respect. Example data indicative of the timing of healthcare visits of the patient 102 include dates of healthcare visits, time intervals between healthcare visits, a number of visits, a duration over which the visits occurred, and/or any other suitable data indicative of visit time, as aspects of the technology described herein are not limited in this respect. The information stored in the at least one healthcare database 110 may be stored in any suitable format and/or using any suitable data structure(s), as aspects of the technology described herein are not limited in this respect. The at least one healthcare database 110 may store data in any suitable way (e.g., one or more databases, one or more files). The at least one database 110 may be a single database or multiple databases.

In some embodiments, server(s) 116 may access information stored in the at least one healthcare database 110 and use this information to perform processes described herein for identifying a patient (e.g., patient 102) for inclusion in a clinical trial.

In some embodiments, server(s) 116 may include one or multiple computing devices. When server(s) 116 include multiple computing devices, the device(s) may be physically co-located (e.g., in a single room) or distributed across multiple physical locations. In some embodiments, server(s) 116 may be part of a cloud computing infrastructure. In some embodiments, one or more server(s) 116 may be co-located in a facility operated by an entity (e.g., a hospital, research institution).

As shown in FIG. 1, in some embodiments, the results of the analysis performed by the server(s) 116 may be provided to one or more users (e.g., user 112) through the healthcare facility computing device 106 and/or remote computing device 114, either of which may be a portable computing device, such as a laptop or smartphone, or a fixed computing device such as a desktop computer. The results may be provided in a written report, an e-mail, a graphical user interface, and/or in any other suitable way. In some embodiments, the results may be provided to a doctor, to a remote user 112, to a person conducting research, to a person conducting a clinical trial, and/or any other suitable user, as aspects of the technology described herein are not limited in this respect.

In some embodiments, the results may be part of a graphical user interface (GUI) presented to a user (e.g., remote user 112). In some embodiments, the GUI may be presented to the user as part of a webpage displayed by a web browser executing on the healthcare facility computing device 106 and/or the remote computing device 114. In some embodiments, the GUI may be presented to the user using an application program (different from a web browser) executing on the healthcare facility computing device 106 and/or the remote computing device 114. For example, in some embodiments, the healthcare facility computing device 106 and/or the remote computing device 114 may be a mobile device (e.g., a smartphone) and the GUI may be presented to the user via an application program (e.g., an app) executing on the mobile device.

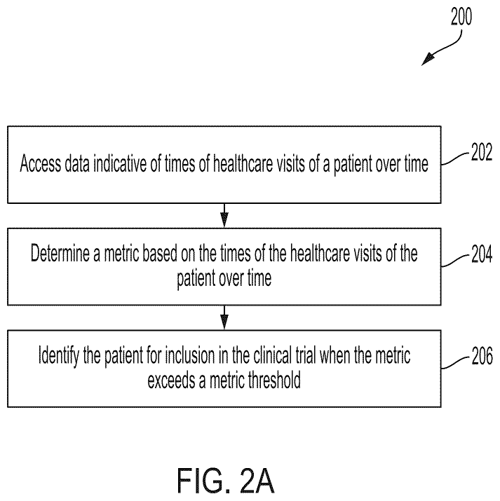

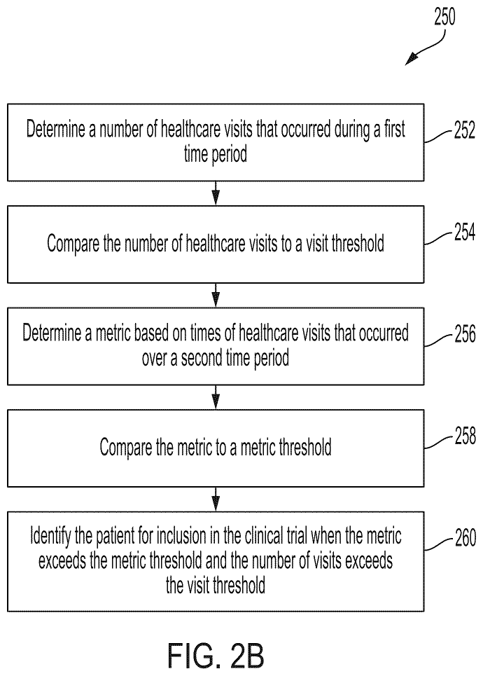

FIG. 2A is a diagram showing an exemplary computerized method 200 for identifying a patient for inclusion in a clinical trial, according to some embodiments. Method 200 may be implemented on any one or more of healthcare facility computing device 106, remote computing device 114, and/or server(s) 116, whether working independently or in cooperation with one another. At step 202, the computing device accesses data indicative of times of healthcare visits of a patient over time. In some embodiments, the data may include dates of healthcare visits, time intervals between healthcare visits, a number of visits, a duration over which the visits occurred, or any other suitable data related to the timing of such visits, as aspects of the technology are not limited in this respect. In some embodiments, the data is obtained for a specified duration of time. For example, the computing device may access data indicative of times of a patient's healthcare visits that occurred during the past two years, one year, six months, three months, one month, one week, or over any other suitable duration of time, as aspects of the technology described herein are not limited in this respect. In some embodiments, the computing device accesses the data from a database, such as the healthcare database 110, for example.

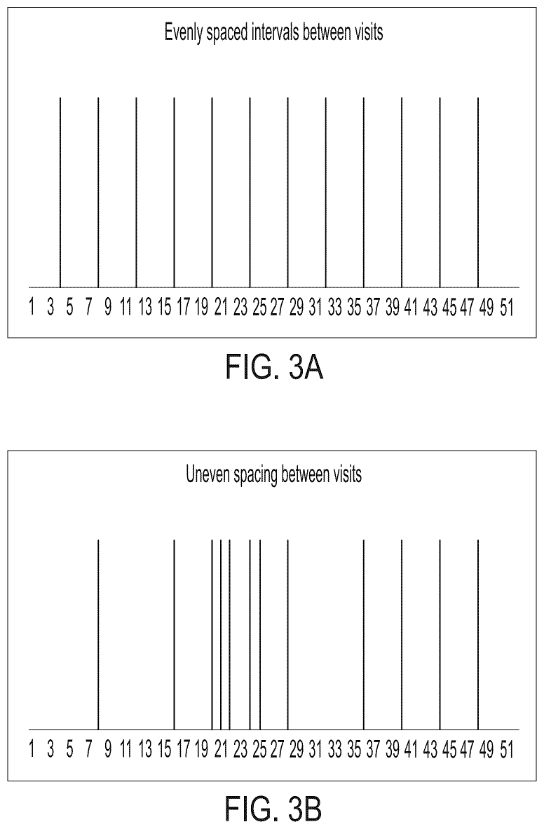

At step 204, the computing device determines a metric based on the times of the healthcare visits of the patient accessed at step 202. In some embodiments, the metric may be indicative of a degree of irregularity (or regularity) of the spacing of the healthcare visits over time. For example, FIGS. 3A-B show charts illustrating the spacing of healthcare visits over time. FIG. 3A shows healthcare visits of a patient that are spaced at relatively regular intervals over the course of the year. In some embodiments, such regularly spaced visits could indicate that the patient is visiting the healthcare facility according to a planned treatment or medical exam schedule. For example, a patient diagnosed with cancer may visit a healthcare facility every week, over the course of a year, to receive chemotherapy. However, if the patient experiences new or worsening symptoms, the periodicity of visits may be interrupted, resulting in visits to the healthcare facility that are random, or less regular. For example, FIG. 3B shows healthcare visits of a patient that are spaced at relatively irregular intervals over the course of the year.

According to some embodiments, determining the metric at step 204 includes comparing the pattern of a patient's healthcare visits to a baseline pattern of visits. For example, if the patient visited the healthcare facility 12 times over the course of a year, the pattern of those visits may be compared to the pattern of 12 evenly-spaced visits over the course of the year. This may include, in some embodiments, applying an analysis function, such as a non-linear compression function, to the time intervals between the healthcare visits of the patient and to the time intervals between the evenly-spaced healthcare visits. Examples of such analysis functions include a logarithm function, a square root function, a cube root function, an inverse function, an arcsine function, a Box-Cox function, and/or any other suitable non-linear compression function, as aspects of the technology described herein are not limited in this respect.

Equation 1 shows an illustrative and non-limiting example of applying such a non-linear compression function to determine the metric, referred to as the Clustered Care Statistic (CCS) in this example.

$$CCI = nT ( \frac{{\sum }_{i = 1}^{n} {d}_{i}}{n} ) / {\sum }_{i = 1}^{n} T ( {d}_{i} ) \qquad Equation 1$$

Where:
T( ) serves as a non-linear compression function;
n represents the number of healthcare visits; and
dᵢ represents the time interval between i'th healthcare visit and the previous (i.e., i−1) healthcare visit, or in the case of i=1, the time interval between the beginning of the observation time period and the first healthcare visit (e.g., measured by a number of days, weeks, months, or any other suitable metric of time).

The numerator of Equation 1 represents the result of applying the non-linear compression function T to n evenly-spaced intervals throughout the time period starting with the beginning of the observation time period and ending with the last healthcare visit, then summing the results, while the denominator of Equation 1 represents the result of applying the non-linear compression function T to each of the actual time intervals dᵢ, between n healthcare visits, then summing the results.

In some embodiments, a generalized CCS optionally includes a visit interval from the last visit, n, to the end of the observation time window. This spacing is denoted as dₙ₊₁. The usage of this additional visit is indicated by a weight, w (0 or 1), resulting in:

$$CCI ( w ) = ( n + w ) T ( \frac{{\sum }_{i = 1}^{n + w} {d}_{i}}{n + w} ) / {\sum }_{i = 1}^{n + w} T ( {d}_{i} ) \qquad Equation 2$$

When w=1, this gives the ratio of the compressed average spacing over an entire observation window to the average of the compressed spacings between visits and the end of the observation period. When w=0, this is the CCS as previously defined above.

In yet further embodiments, a CCS may further take into account not only the intervals dᵢ, between healthcare visits, but also the recency of each healthcare visit. For example, a CCS may be modified to discount (or weigh less heavily) events that happened a long time ago. One example of a CCS according to this embodiment is provided below:

$$Equation 3$$

$$CCI ( w ) = ( n + w ) T ( \frac{{\sum }_{i = 1}^{n + w} {d}_{i} * f ( {t}_{n + w} - {t}_{i} )}{n + w} ) / {\sum }_{i = 1}^{n + w} T ( {d}_{i} * f ( {t}_{n + w} - {t}_{i} ) )$$

Where:
T( ), dᵢ, n, and w are as previously defined, and
tᵢ, represents the time (e.g., date and/or time of day) of the i'th healthcare visit, and
f( ) represents a function where f(0)=1 and f(x) monotonically decays to zero, or something between 0 and 1.

In this way, the CCS may discount healthcare visits that happened a long time ago, and place relatively greater weight on visits that happened more recently.

At step 206, the computing device compares the metric determined at step 204 to a threshold and identifies the patient for inclusion in a clinical trial when the metric exceeds the threshold. According to some embodiments, in determining the metric, the non-linear compression function may compress larger values more than it compresses smaller values. Because irregularly spaced healthcare visits introduce longer time intervals between healthcare visits (e.g., as opposed to if the visits were evenly spaced), the sum of the non-linearly compressed values will be smaller than if those visits were regularly spaced. Therefore, the result of Equation 1, 2, and/or 3 may be relatively large when the healthcare visits are more irregularly spaced (e.g., introducing greater time intervals between visits), whereas the result may be relatively small (e.g., close to 1) when the healthcare visits are more regularly spaced. If the metric is sufficiently large (e.g., greater than the threshold), then the patient may be identified for inclusion in the clinical trial. As described herein above, healthcare visits that are irregularly spaced may indicate that the patient experiences disease flares. As a result, the patient may qualify for inclusion in a clinical trial.

FIG. 2B is a diagram showing an exemplary computerized method 250 for identifying a patient for inclusion in a clinical trial, according to some embodiments. Prior to process 250, the computing device may access data indicative of times of healthcare visits of a patient. In some embodiments, accessing the data may include performing the techniques described herein including at least with respect to step 202 of FIG. 2A.

At step 252, the computing device determines a number of healthcare visits that occurred during a first time period. For example, the first time period may include the preceding day(s), the preceding week(s), the preceding month(s), or the preceding year(s).

At step 254, the computing device compares the determined number of healthcare visits to a visit threshold. In some embodiments, if the number of visits exceeds the threshold, this may indicate that the patient has had an increased number of visits compared to what is normal for that patient. Therefore, in some embodiments, the visit threshold may depend upon the patient (e.g., the patient's normal healthcare visit schedule) and/or on the duration of time over which the number of healthcare visits occurred. For example, two healthcare visits over the period of two weeks may be normal for some patients, while it may be more unusual for others. Similarly, three healthcare visits over the course of a year may be normal for a patient, while three healthcare visits over the course of three weeks may be unusual for that same patient.

At step 256, the computing device determines a metric based on the times of healthcare visits that occurred over a second time period. According to some embodiments, determining the metric may include performing the techniques described herein including at least with respect to step 204 of FIG. 2A. In some embodiments, the determined metric may be indicative of a degree of irregularity (or regularity) of the spacing of healthcare visits over the second time period. According to some embodiments, the second time period may include the preceding day(s), the preceding week(s), the preceding month(s), or the preceding year(s). The second time period, in some embodiments, may be longer than the first time period described with respect to step 252. For example, the first time period may include the preceding few weeks, while the second time period includes the preceding year.

At step 258, the computing device compares the metric to a metric threshold, as described herein including at least with respect to step 206 of FIG. 2A.

At step 260, the computing device identifies the patient for inclusion in the clinical trial when the metric exceeds the metric threshold and when the number of visits exceeds the visit threshold.

Referring to steps 252-258 of process 250, it should be appreciated that steps 252 and 254 may be performed before, after, or concurrently with steps 256 and 258, as aspects of the technology described herein are not limited in this respect. For example, the computing device may determine and compare the number of healthcare visits to the visit threshold before, after, or at the same time as it determines and compares the metric to the metric threshold. In some embodiments, the output of step 254 may inform whether or not the computing device continues to perform process 250. For example, if the computing device determines, at step 254, that the number of healthcare visits does not exceed the visit threshold, then process 250 may end. In some embodiments, the output of step 258 may inform whether or not the computing device continues to perform process 250. For example, if the computing device determines at step 258 that the metric does not exceed the metric threshold, then process 250 may end.

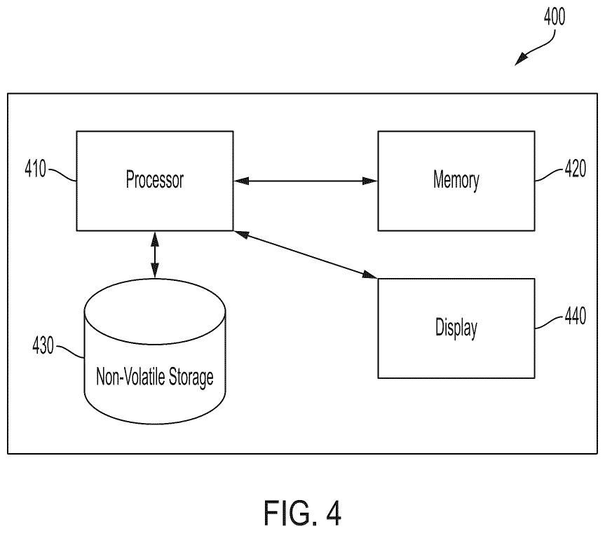

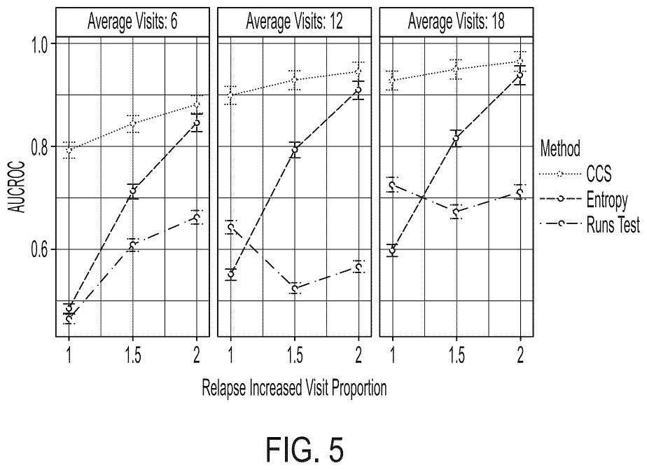

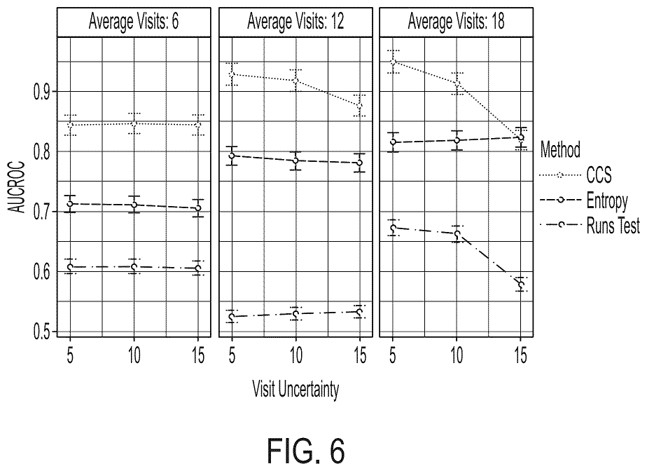

An illustrative implementation of a computer system 400 that may be used to perform any of the aspects of the techniques and embodiments disclosed herein is shown in FIG. 4. The computer system 400 may include one or more processors 410 and one or more non-transitory computer-readable storage media (e.g., memory 420 and one or more non-volatile storage media 430) and a display 440. The processor 410 may control writing data to and reading data from the memory 420 and the non-volatile storage device 430 in any suitable manner, as the aspects of the invention described herein are not limited in this respect. To perform functionality and/or techniques described herein, the processor 410 may execute one or more instructions stored in one or more computer-readable storage media (e.g., the memory 420, storage media, etc.), which may serve as non-transitory computer-readable storage media storing instructions for execution by the processor 410.

In connection with techniques described herein, code used to, for example, identify a patient for inclusion in a clinical trial may be stored on one or more computer-readable storage media of computer system 400. Processor 410 may execute any such code to provide any techniques for recognizing objects as described herein. Any other software, programs or instructions described herein may also be stored and executed by computer system 400. It will be appreciated that computer code may be applied to any aspects of methods and techniques described herein. For example, computer code may be applied to interact with an operating system to recognize objects through conventional operating system processes.

### EXAMPLES

In one example, a simulation study was used to explore the properties of the CCS and compare the performance of the CCS to sample entropy (SE) and the Wald-Wolfowitz runs statistic in detecting patients with irregular visits. All simulations were performed using the R language (version 4.1.2). Sample entropy was calculated using the R package, pracma, via the sample entropy function (see Borchers H W. pracma: Practical Numerical Math Functions 2022. Available from: https://CRAN.R-project.org/package=pracma). The Wald-Wolfowitz runs statistic was calculated using the R package, randtests using the runs.test function (see Caeiro F, Mateus A. randtests: Testing Randomness in R. 2022. Available from: https://CRAN.R-project.org/package=randtests).

To evaluate the performance of the methods, the following approach was used to generate patients across a parameterized spectrum from regular to irregular visit patterns. The approach enables the definition of a “relapsed” patient, one with visits that are more random, frequent, or clustered, that our method intends to detect, versus the reference patients, receiving more optimized and regular care. The reference patient's arrival rate pattern ranges from completely regular visits, i.e., where the inter-visit time is constant (e.g. every 4 weeks), to completely random visits, where the inter-visit times have an exponential distribution. This enables modulation of the difficulty in determining relapsed from reference patients in the presence of increasing or decreasing background variability.

For each scenario, all patients were set to have an average number of visits, V , over the entire time window, N. For a completely regular visit pattern, visits are expected to arrive at intervals of N/V , with the timing of the initial visit sampled as,

$${v}_{0} ∼ Uniform ( 1 , \frac{N}{\overline{V}} ) .$$

Since completely regular visits are not observed in practice, uncertainty on the planned arrival time for visit i was simulated as

$${v}_{i} ∼ {v}_{0} + i \frac{N}{\overline{V}} + Uniform ( - {σ}_{V} , {σ}_{V} ) .$$

By increasing the parameter, σ_{V}, the degree of regularity decreases. All patients simulated with this approach are considered as having regular visits, and an effective algorithm should not flag them as having an irregular pattern.

The patients that should be detected are “relapsed” or those with irregular visits beyond the expected uncertainty provided by σ_{V}. To generate these patients, a probability of relapse, p_{R}, is used. For those selected as relapsed, the time of relapse, N_{R}, is drawn from a uniform distribution over the entire time window as, N_{R}˜Uniform(1, N). For visits prior to N_{R}, patients are simulated using regular arrivals as previously defined, and after N_{R} patients have exponentially distributed inter-arrival times (random arrivals) over the interval with an expected frequency, f_{R}N/V̅, where f_{R} indicates the degree to which the expected frequency changes during relapse. For values greater than 1, this indicates that patient arrivals are both irregular and more frequent. Each method was then evaluated on the ability to distinguish between regular and relapsed patients in the dataset, i.e., whether the patient is in relapse (has a disease flare) or not.

To evaluate the performance of CCS versus entropy and the runs test, a range of scenarios were constructed using the parameters defined in the data generation section. A full factorial simulation experiment for 3 factors was performed, 1) V̅, the regular visit frequency (6, 12, and 18 visits per year), 2) σ_{V}, the degree to which a visit may depart from the expected date and still be counted as ‘regular’ (5, 10, 15 days), and 3) the increase in proportion of visits during relapse, f_{R}, (1, 1.5, 2—indicating the same frequency, a 50% increase, or a 100% increase in the number of visits during relapse). For all scenarios, N was set to 365 (daily visits over a year), and p_{R} was set to 0.1.

In total there were 27 scenarios were simulated (three factors varied, each with 3 settings). For each scenario, a random number generator seed was set and 1,000 virtual patients were generated a total of 100 times for each scenario. Simulations were performed on a Linux cluster, distributed across 270 cores.

To compare performance of CCS, entropy, and the runs statistic in classifying relapsed patients, the area under the receiver operator curve (AUCROC) was used. For each scenario the 10,000 patients were evaluated using the R package, pROC, to calculate a ROC curve (and resulting AUCROC) for a set of thresholds across each statistic (see Robin X, Turck N, Hainard A, Tiberti N, Lisacek F, Sanchez J C, et al. pROC: Display and Analyze ROC Curves. 2021. Available from: https://CRAN.R-project.org/package=pROC). The AUCROC values were summarized in tabular form and visualized to compare and contrast the methods across scenarios.

For each of the 27 simulation scenarios, the visit pattern over 1 year for 1,000 virtual patients were simulated 100 times. Each visit pattern was assessed using the CCS and sample entropy statistic and a cutoff was evaluated for each score to determine if a patient was in relapse. The average AUCROC and standard error (SE) across simulations results for each scenario are provided in table 1.

TABLE 1
Average AUCROC and standard error (SE) across simulations for
the scenarios comparing CCS, entropy, and the runs statistic.

| | | Increase in Visit | | | |
|---|---|---|---|---|---|
| Number of | Visit | Frequency after | CCS - AUCROC | Entropy - | Runs Statistic - |
| Regular Visits | Uncertainty | Relapse | (SE) | AUCROC (SE) | AUCROC (SE) |
| 6 | 5 | 1 | 0.793 (0.008) | 0.485 (0.005) | 0.465 (0.005) |
| 12 | 5 | 1 | 0.899 (0.009) | 0.550 (0.006) | 0.643 (0.006) |
| 18 | 5 | 1 | 0.928 (0.009) | 0.598 (0.006) | 0.726 (0.007) |
| 6 | 10 | 1 | 0.795 (0.008) | 0.495 (0.005) | 0.498 (0.005) |
| 12 | 10 | 1 | 0.881 (0.009) | 0.548 (0.005) | 0.635 (0.006) |
| 18 | 10 | 1 | 0.867 (0.009) | 0.598 (0.006) | 0.719 (0.007) |
| 6 | 15 | 1 | 0.790 (0.008) | 0.497 (0.005) | 0.504 (0.005) |
| 12 | 15 | 1 | 0.820 (0.008) | 0.562 (0.006) | 0.648 (0.006) |
| 18 | 15 | 1 | 0.743 (0.007) | 0.551 (0.006) | 0.602 (0.006) |
| 6 | 5 | 1.5 | 0.844 (0.008) | 0.712 (0.007) | 0.609 (0.006) |
| 12 | 5 | 1.5 | 0.929 (0.009) | 0.793 (0.008) | 0.525 (0.005) |
| 18 | 5 | 1.5 | 0.950 (0.009) | 0.815 (0.008) | 0.673 (0.007) |
| 6 | 10 | 1.5 | 0.847 (0.008) | 0.712 (0.007) | 0.609 (0.006) |
| 12 | 10 | 1.5 | 0.918 (0.009) | 0.784 (0.008) | 0.530 (0.005) |
| 18 | 10 | 1.5 | 0.913 (0.009) | 0.818 (0.008) | 0.662 (0.007) |
| 6 | 15 | 1.5 | 0.844 (0.008) | 0.705 (0.007) | 0.606 (0.006) |
| 12 | 15 | 1.5 | 0.877 (0.009) | 0.781 (0.008) | 0.533 (0.005) |
| 18 | 15 | 1.5 | 0.819 (0.008) | 0.824 (0.008) | 0.579 (0.006) |
| 6 | 5 | 2 | 0.881 (0.009) | 0.846 (0.008) | 0.662 (0.007) |
| 12 | 5 | 2 | 0.945 (0.009) | 0.909 (0.009) | 0.567 (0.006) |
| 18 | 5 | 2 | 0.965 (0.010) | 0.938 (0.009) | 0.711 (0.007) |
| 6 | 10 | 2 | 0.883 (0.009) | 0.841 (0.008) | 0.653 (0.007) |
| 12 | 10 | 2 | 0.940 (0.009) | 0.911 (0.009) | 0.562 (0.006) |
| 18 | 10 | 2 | 0.941 (0.009) | 0.936 (0.009) | 0.710 (0.007) |
| 6 | 15 | 2 | 0.884 (0.009) | 0.844 (0.008) | 0.653 (0.007) |
| 12 | 15 | 2 | 0.923 (0.009) | 0.916 (0.009) | 0.557 (0.006) |
| 18 | 15 | 2 | 0.880 (0.009) | 0.928 (0.009) | 0.619 (0.006) |

Across the simulation scenarios, the CCS method had a higher AUCROC relative to entropy, indicating improved performance in detecting relapse. The degree of benefit of CCS over entropy was influenced by the frequency of regular visits as well as the increased rate of visits during relapse. FIG. 5 shows the performance on AUCROC of CCS, Entropy, and Runs test as a function of the Relapse Increased Visit Proportion and Average Visits (for visit uncertainty of 5 days). As shown in FIG. 5, the AUCROC for CCS is higher than entropy but has a greater degree of improvement for when the increase in frequency of visits during relapse is less. Also, overall, the AUCROC for both methods is higher as the average visit frequency increases. This indicates that the CCS is more substantially sensitive than entropy in case where the primary difference is driven by irregular frequency versus irregular and increased frequency. For example, once the rate of difference in frequency approaches 2×, the methods preform similarly on AUCROC.

FIG. 6 shows the performance on AUCROC of CCS, Entropy, and Runs test as a function of the Visit Uncertainty and Average Visits (for relapse visit proportion of 1.5). This figure illustrates the impact of visit uncertainty on the relative performance of the methods. Across the scenarios considered, the CCS generally performs best compared to other methods, with a decrease in performance relative to entropy for 18 average visits with an uncertainty of 15 days. For this setting, the distribution of visits for non-relapsed patients is nearly uniform masking the departure from regularity in the relapsed period.

In addition to the improved AUCROC for the CCS approach versus entropy, CCS met its objective of being a simple and computationally efficient tool to identify visit irregularity. A benchmark comparison of the speed to compute CCS versus entropy for a single visit schedule, demonstrated that CCS is 211 times faster than entropy, averaging 1.1 ms vs 221.7 ms for the sample entropy statistic. For large datasets, this reduced computational burden is also a key differentiator.

The CCS could be a useful tool that has multiple potential applications that broadly fall into three categories: 1) Health Economics and Outcomes Research. Here the CCS can quantify care delivery on the important dimension of regularity both at the population level as well as for the individual patient. Irregular care has been associated with poor health outcomes and monitoring this metric could be used to prioritize interventions where they are most needed. 2) Clinical Trial Recruitment. Finding the appropriate patients for clinical trials is expensive and time consuming—and one of the main drivers of the overall trial duration. The CCS could be used, as explored in this simulation model, as an electronic health record tool to detect recent bursts of healthcare visits, that given the previously established pattern for the patient and the chronic disease, is unusual. Associated patient records could be flagged for review in the EMIR to determine whether the patient is indeed having such a flare and if otherwise eligible, be offered clinical trial participation. Not only would CCS reduce recruitment costs, but it would also ensure that the right patients are recruited during the appropriate stage of the disease, thereby increasing the statistical power of the trial. 3) Outbreak detection. Here urgent care and emergency visits could be tracked, and the CCS could support other established syndromic surveillance methods that are largely borrowed from process control such as CUSUM-based methodologies (18).

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This allows elements to optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

Various aspects are described in this disclosure, which include, but are not limited to, the following aspects:

1. A computerized method for identifying a patient for inclusion in a clinical trial, the method comprising:
accessing data indicative of times of a plurality of healthcare visits of the patient over time during an observation period;
determining a metric based on the times of the plurality of healthcare visits of the patient over time during the observation period; and identifying the patient for inclusion in the clinical trial when the metric exceeds a metric threshold.
   2. The computerized method of aspect 1, wherein the metric is indicative of a pattern of occurrence of the plurality of healthcare visits over time during the observation period.
   3. The computerized method of any of aspects 1-2, wherein determining the metric comprises analyzing a spacing of the plurality of healthcare visits over time during the observation period.
   4. The computerized method of any of aspects 1-3, wherein determining the metric comprises determining a degree to which the plurality of healthcare visits are irregularly spaced over time during the observation period.
   5. The computerized method of aspects 1-4, wherein determining the metric comprises applying a non-linear compression function to a plurality of time intervals, the plurality of time intervals comprising at least each time interval between the plurality of healthcare visits, wherein the non-linear compression function compresses larger values more than smaller values.
   6. The computerized method of aspect 5, wherein the plurality of time intervals further comprises a beginning time interval between a beginning of the observation period and a first healthcare visit of the plurality of healthcare visits.
   7. The computerized method of aspect 6, wherein the plurality of time intervals further comprises an ending time interval between a last healthcare visit of the plurality of healthcare visits and an ending of the observation period.
   8. The computerized method of any of aspects 5-7, wherein determining the metric comprises determining a first value by summing the compressed plurality of time intervals.
   9. The computerized method of any of aspects 5-7, wherein determining the metric comprises determining a first value by summing the compressed plurality of time intervals, wherein time intervals between healthcare visits that occurred more recently in time are accorded greater weight than time intervals between healthcare visits that occurred less recently in time.
   10. The computerized method of any of aspects 5-9, wherein determining the metric comprises determining a second value by applying the non-linear compression function to a quotient of (i) a sum of the plurality of time intervals and (ii) a number of time intervals.
   11. The computerized method of any of aspects 5-9, wherein determining the metric comprises determining a second value by applying the non-linear compression function to a quotient of (i) a sum of the plurality of time intervals, wherein time intervals between healthcare visits that occurred more recently in time are accorded greater weight than time intervals between healthcare visits that occurred less recently in time and (ii) a number of time intervals.
   12. The computerized method of any of aspects 10-11, wherein determining the metric comprises multiplying the second value by the number of time intervals to generate a third value.
   13. The computerized method of aspect 12, wherein determining the metric comprises dividing the third value by the first value.
   14. The computerized method of any of aspects 5-13, wherein the non-linear compression function includes at least one of a logarithm function, a square root function, a cube root function, an inverse function, an arcsine function, and a Box-Cox function.
   15. The computerized method of any of aspects 1-14, further comprising:
determining a number of the plurality of healthcare visits that occurred over a second time period;
determining whether the number of the plurality of healthcare visits exceeds a visit threshold; and
identifying the patient for inclusion in the clinical trial when the number of the plurality of healthcare visits exceeds the visit threshold and the metric exceeds the metric threshold.
   16. The computerized method of aspect 15, wherein the second time period is shorter than the observation period.
   17. The computerized method of any of aspects 1-16, wherein the metric is indicative of a disease flare of the disease of the patient.
   18. A non-transitory computer-readable storage media comprising instructions that, when executed by one or more processors on a computing device, are operable to cause the one or more processors to execute the method of any of aspects 1-17.
   19. A system comprising a memory storing instructions, and a processor configured to execute the instructions to perform the method of any of aspects 1-17.

1. A computerized method for identifying subjects for inclusion in a clinical trial, the method comprising:
accessing data indicative of times of a plurality of healthcare visits of a patient over time during an observation period;
determining a metric based on the times of the plurality of healthcare visits of the patient over time during the observation period; and
identifying the patient for inclusion in the clinical trial when the metric exceeds a metric threshold.

2. The computerized method of claim 1, wherein the metric is indicative of a pattern of occurrence of the plurality of healthcare visits over time during the observation period.

3. The computerized method of claim 1, wherein determining the metric comprises analyzing a spacing of the plurality of healthcare visits over time during the observation period.

4. The computerized method of claim 1, wherein determining the metric comprises determining a degree to which the plurality of healthcare visits are irregularly spaced over time during the observation period.

5. The computerized method of claim 1, wherein determining the metric comprises applying a non-linear compression function to a plurality of time intervals, the plurality of time intervals comprising at least each time interval between the plurality of healthcare visits, wherein the non-linear compression function compresses larger values more than smaller values.

6. The computerized method of claim 5, wherein the plurality of time intervals further comprises a beginning time interval between a beginning of the observation period and a first healthcare visit of the plurality of healthcare visits.

7. The computerized method of claim 6, wherein the plurality of time intervals further comprises an ending time interval between a last healthcare visit of the plurality of healthcare visits and an ending of the observation period.

8. The computerized method of claim 5, wherein determining the metric comprises determining a first value by summing the compressed plurality of time intervals.

9. The computerized method of claim 5, wherein determining the metric comprises determining a first value by summing the compressed plurality of time intervals, wherein time intervals between healthcare visits that occurred more recently in time are accorded greater weight than time intervals between healthcare visits that occurred less recently in time.

10. The computerized method of claim 5, wherein determining the metric comprises determining a second value by applying the non-linear compression function to a quotient of (i) a sum of the plurality of time intervals and (ii) a number of time intervals.

11. The computerized method of claim 5, wherein determining the metric comprises determining a second value by applying the non-linear compression function to a quotient of (i) a sum of the plurality of time intervals, wherein time intervals between healthcare visits that occurred more recently in time are accorded greater weight than time intervals between healthcare visits that occurred less recently in time and (ii) a number of time intervals.

12. The computerized method of claim 10, wherein determining the metric comprises multiplying the second value by the number of time intervals to generate a third value.

13. The computerized method of claim 12, wherein determining the metric comprises dividing the third value by the first value.

14. The computerized method of claim 5, wherein the non-linear compression function includes at least one of a logarithm function, a square root function, a cube root function, an inverse function, an arcsine function, and a Box-Cox function.

15. The computerized method of claim 1, further comprising:
determining a number of the plurality of healthcare visits that occurred over a second time period;
determining whether the number of the plurality of healthcare visits exceeds a visit threshold; and
identifying the patient for inclusion in the clinical trial when the number of the plurality of healthcare visits exceeds the visit threshold and the metric exceeds the metric threshold.

16. The computerized method of claim 15, wherein the second time period is shorter than the observation period.

17. The computerized method of claim 1, wherein the metric is indicative of a disease flare of a disease of the patient.

18. (canceled)

19. (canceled)
